(19) United States
(12) Patent Application Publication
(10) Pub. No.: US 2026/0225078 A1
(43) Pub. Date: Aug. 6, 2026

(54) OXIDATIVE COUPLING CATALYST, METHOD FOR PRODUCING OXIDATIVE COUPLING CATALYST, AND METHOD FOR PRODUCING HYDROCARBONS

(71) Applicants: MITSUBISHI HEAVY INDUSTRIES, LTD., Tokyo (JP); The University of Tokyo, Tokyo (JP)

(72) Inventors: Atsuhiro YUKUMOTO, Tokyo (JP); Yukio TANAKA, Tokyo (JP); Masanao YONEMURA, Tokyo (JP); Koichiro YOSHITOKU, Tokyo (JP); Kazuhiro TAKANABE, Tokyo (JP)

(73) Assignees: MITSUBISHI HEAVY INDUSTRIES, LTD., Tokyo (JP); The University of Tokyo, Tokyo (JP)

(21) Appl. No.: 19/148,064

(22) PCT Filed: Jan. 12, 2024
(86) PCT No.: PCT/JP2024/000668

§ 371 Date: Jul. 15, 2025

(30) Foreign Application Priority Data
Jan. 27, 2023 (JP) 2023-010932

(51) Int. Cl. B01J 23/30; B01J 21/08; B01J 23/04; B01J 23/14; B01J 23/34; B01J 35/80; B01J 37/00; B01J 37/02; B01J 37/08; C07C 2/84

(52) U.S. Cl. CPC B01J 23/30; B01J 21/08; B01J 23/04; B01J 23/14; B01J 23/34; B01J 35/80; B01J 37/0009; B01J 37/0201; B01J 37/08; C07C 2/84; C07C 2521/08; C07C 2523/04; C07C 2523/14; C07C 2523/30; C07C 2523/34

(57) ABSTRACT

The object of the present disclosure is to provide an oxidative coupling catalyst, a method for producing an oxidative coupling catalyst, and a method for producing hydrocarbons, which enable production of a hydrocarbon having 2 or more carbon atoms with a high yield even at a high temperature under pressure and enable size reduction of a facility that carries out an OCM reaction. An aspect of the present invention is an oxidative coupling catalyst generates a hydrocarbon having 2 or more carbon atoms from methane by an oxidative coupling reaction of methane, in which the oxidative coupling catalyst is a sintered substance of silica on which an alkali metal salt is supported.

### TECHNICAL FIELD

The present disclosure relates to an oxidative coupling catalyst, a method for producing an oxidative coupling catalyst, and a method for producing hydrocarbons. Priority is claimed on Japanese Patent Application No. 2023-010932, filed Jan. 27, 2023, the content of which is incorporated herein by reference.

### BACKGROUND ART

A technology for producing a hydrocarbon such as an olefin by an oxidative coupling reaction using a gas containing methane such as a natural gas (oxidative coupling of methane, hereinafter, also referred to as “OCM reaction”) is known.

For example, Patent Document 1 suggests a method for producing a hydrocarbon by carrying out an OCM reaction to generate a hydrocarbon having 2 or more carbon atoms from methane using a catalyst (oxidative coupling catalyst) in which oxides or composite oxides of sodium, manganese, and tungsten are supported by an inorganic oxide. According to the invention of Patent Document 1, an OCM reaction is carried out with high efficiency to produce a hydrocarbon having 2 or more carbon atoms with a high yield.

### CITATION LIST

### Patent Document

Patent Document 1: Japanese Patent No. 5493928

### SUMMARY OF INVENTION

### Technical Problem

However, in the invention of Patent Document 1, the OCM reaction at the atmospheric pressure is targeted, and the reactor that performs the OCM reaction may be enlarged, and an increase in power of a compressor or the like that supplies a process gas to a purification step and a separation step on a downstream of the reactor may occur.

In order to solve such a problem, it is considered that the OCM reaction is carried out at a high temperature under pressure (for example, 750° C., 0.9 MPa). However, in a case where the OCM reaction is carried out at a high temperature under pressure, the active component of the oxidative coupling catalyst is melted, volatilized, and reduced, and thus the yield of the hydrocarbon to be obtained may be reduced.

In addition, as another problem, in a case of a catalyst using an inert silica as a carrier, the catalyst may be broken during sintering of the catalyst in a case where a silica gel having a large particle diameter is used.

The present disclosure has been made in order to solve the above-described problems, and an object thereof is to provide an oxidative coupling catalyst, a method for producing an oxidative coupling catalyst, and a method for producing hydrocarbons, which enable production of a hydrocarbon having 2 or more carbon atoms with a high yield even at a high temperature under pressure and enable size reduction of a facility that carries out an OCM reaction.

### Solution to Problem

In order to solve the above-described problems, an oxidative coupling catalyst according to the present disclosure is an oxidative coupling catalyst that generates a hydrocarbon having 2 or more carbon atoms from methane by an oxidative coupling reaction of methane, which is a sintered substance of silica on which an alkali metal salt is supported.

A method for producing an oxidative coupling catalyst according to the present disclosure is a method for producing an oxidative coupling catalyst that generates a hydrocarbon having 2 or more carbon atoms from methane by an oxidative coupling reaction of methane, the method including: a step of bringing an aqueous solution containing an alkali metal salt into contact with silica or a sintered substance of silica to obtain a precursor in which the silica or the sintered substance of silica is impregnated with the aqueous solution; and a step of sintering the precursor at 800° C. or higher to obtain the oxidative coupling catalyst.

A method for producing hydrocarbons according to the present disclosure is a method of generating a hydrocarbon having 2 or more carbon atoms from methane by an oxidative coupling reaction of methane using the above-described oxidative coupling catalyst.

### Advantageous Effects of Invention

According to the present disclosure, it is possible to produce a hydrocarbon having 2 or more carbon atoms with a high yield even at a high temperature under pressure and to reduce the size of a facility that performs an OCM reaction.

### BRIEF DESCRIPTION OF DRAWINGS

FIG. 1 A flowchart showing a method for producing an oxidative coupling catalyst according to an embodiment of the present disclosure.

FIG. 2 A flowchart showing a method for producing hydrocarbons according to an embodiment of the present disclosure.

FIG. 3 A photograph showing silica gel particles after sintering.

FIG. 4 A photograph showing extrusion-molded silica after sintering.

FIG. 5 A graph showing results of X-ray diffraction (XRD) of extrusion-molded silica after sintering.

FIG. 6 A graph showing results of X-ray diffraction (XRD) of an oxidative coupling catalyst according to an embodiment of the present disclosure.

FIG. 7 A flowchart showing a method for producing an oxidative coupling catalyst according to Example 1.

FIG. 8 A flowchart showing a method for producing an oxidative coupling catalyst according to Example 2.

FIG. 9 A flowchart showing a method for producing an oxidative coupling catalyst according to Example 3.

FIG. 10 A photograph showing an oxidative coupling catalyst obtained in Example 3.

FIG. 11 A graph showing a yield of an olefin having 2 to 4 carbon atoms in a generated gas, a conversion rate of methane, a selectivity of a hydrocarbon having 2 to 4 carbon atoms, and an oxygen consumption rate with respect to a partial pressure of methane in a raw material gas in a case where an oxidative coupling reaction is carried out using the oxidative coupling catalysts obtained in Examples 2, 4, and 5.

FIG. 12 A graph showing a conversion rate of methane in a case where an oxidative coupling reaction of methane is carried out using the oxidative coupling catalyst according to the embodiment of the present disclosure.

FIG. 13 A graph showing an oxygen consumption rate in a case where an oxidative coupling reaction of methane is carried out using the oxidative coupling catalyst according to the embodiment of the present disclosure.

### DESCRIPTION OF EMBODIMENTS

### <<Oxidative Coupling Catalyst>>

An oxidative coupling catalyst according to an embodiment of the present disclosure is a sintered substance of silica on which an alkali metal salt is supported.

In the present specification, “sintered substance” denotes a porous body obtained by kneading silica powder together with a binder and performing a heat treatment on the silica molded by a method such as extrusion molding at 700° C. or higher.

The particle diameter of the oxidative coupling catalyst of the present embodiment is preferably 1 to 5 mm and more preferably 2 to 4 mm. In a case where the particle diameter of the oxidative coupling catalyst is large, the pressure loss of a catalyst layer in a case where the OCM reaction is carried out under a pressurized condition can be further reduced, but the catalyst surface area per unit volume of the catalyst layer is reduced, and a large amount of catalyst is required to exhibit predetermined performance. On the contrary, in a case where the particle diameter of the oxidative coupling catalyst is small, the catalyst surface area per unit volume of the catalyst layer is increased, and predetermined performance can be exhibited with a small amount of the catalyst, but the pressure loss of the catalyst layer is increased. In a facility (OCM plant) that performs the OCM reaction, an increase in pressure of a process gas is performed by a compressor or the like on a downstream of an OCM reactor, and a decrease in gas pressure due to the pressure loss of the catalyst layer has a significant effect on the power of the compressor or the like. Therefore, in a case where the particle diameter of the oxidative coupling catalyst is set to be in the above-described numerical ranges, the amount of the catalyst and the pressure loss can be set to be optimum ranges. The particle diameter can be measured, for example, by a sieving method using several kinds of sieves having different openings.

In a case where the particle diameter of the oxidative coupling catalyst varies, the evaluation is carried out based on the average particle diameter. The average particle diameter of the oxidative coupling catalyst of the present embodiment is preferably 1 to 5 mm and more preferably 2 to 4 mm. The average particle diameter is obtained by, for example, a median diameter (d50) of a plurality of particle diameters as measurement results measured by a method such as image analysis.

From the viewpoint of further enhancing the catalytic activity, it is preferable that the oxidative coupling catalyst of the present embodiment exhibits crystallinity.

Whether or not the oxidative coupling catalyst exhibits crystallinity can be confirmed by X-ray diffraction (XRD). In XRD, in a case where a peak is observed at a specific diffraction angle, that is, in a case where a diffraction phenomenon is observed, it is determined that the oxidative coupling catalyst exhibits crystallinity.

Examples of an alkali metal salt which is an active component of the oxidative coupling catalyst according to the present embodiment include an alkali metal salt of an oxide containing at least tungsten or zirconium (hereinafter, also simply referred to as “oxide”).

Examples of the oxide containing at least tungsten or zirconium include tungsten oxide (VI), tungstic acid, zirconia (ZrO₂), and zircon (ZrSiO₄). The oxide containing at least tungsten or zirconium may be a composite oxide of a metal element other than tungsten and zirconium, and tungsten or zirconium.

The metal element other than tungsten and zirconium is not particularly limited, and examples thereof include aluminum, magnesium, calcium, titanium, chromium, manganese, iron, cobalt, nickel, copper, zinc, palladium, silver, indium, tin, iridium, platinum, and gold. These metal elements may or may not form a composite oxide with tungsten or zirconium. Among these metal elements, manganese, indium, and tin are preferable from the viewpoint that the yield of a hydrocarbon having 2 to 4 carbon atoms in the OCM reaction can be further increased, and tin is more preferable from the viewpoint that the selectivity of the hydrocarbon having 2 to 4 carbon atoms can be further increased even under a pressurized condition.

Examples of the alkali metal in the alkali metal salt include sodium, potassium, rubidium, and cesium. Among these alkali metals, potassium is preferable from the viewpoint of high catalytic activity and excellent heat resistance. This is because potassium tungstate (K₂WO₄, melting point: 921° C.) does not melt and the durability is excellent in a case where the operation temperature of the reactor is lower than 900° C., whereas sodium tungstate (Na₂WO₄, melting point: 698° C.) melts and the durability is degraded in a case where the operation temperature of the reactor is extremely high.

In a case where the oxidative coupling catalyst of the present embodiment contains manganese and a potassium salt of an oxide, the mass ratio represented by manganese:potassium salt of oxide:silica is preferably (0.01 to 5):(0.05 to 10):(85 to 99.9), more preferably (0.5 to 4):(1 to 8):(88 to 98.5), and still more preferably (1 to 3):(2 to 7):(90 to 97). Here, the total mass of the manganese, the potassium salt of an oxide, and the silica is set to 100. In a case where the mass ratio represented by manganese:potassium salt of oxide:silica is in the above-described numerical ranges, the selectivity of the hydrocarbon having 2 to 4 carbon atoms can be further increased in the OCM reaction even under a pressurized condition.

The mass ratio (mass concentration) of manganese and potassium in the mass ratio represented by manganese:potassium salt of oxide:silica is determined by analyzing the oxidative coupling catalyst using inductively coupled plasma atomic emission spectroscopy (ICP-AES). The mass ratio (mass concentration) of silica can be calculated by subtracting the mass ratio (mass concentration) of each active component (manganese and potassium) from the total mass of the oxidative coupling catalyst.

The mass ratio represented by manganese:potassium salt of oxide:silica can be adjusted by the concentration of an aqueous solution of each raw material, the amount thereof, and a combination thereof.

In a case where the oxidative coupling catalyst of the present embodiment contains tin, manganese, and a potassium salt of an oxide, the mass ratio represented by tin:manganese:potassium salt of oxide:silica is preferably (0.01 to 50):(0.01 to 5):(0.05 to 10):(35 to 99.9), more preferably (2 to 50):(0.5 to 4):(1 to 8):(38 to 96.5), and still more preferably (10 to 20):(1 to 3):(2 to 7):(70 to 87). Here, the total mass of the tin, the manganese, the potassium salt of an oxide, and the silica is set to 100. In a case where the mass ratio represented by tin:manganese:potassium salt of oxide:silica is in the above-described numerical ranges, the selectivity of the hydrocarbon having 2 to 4 carbon atoms can be further increased in the OCM reaction even under a pressurized condition.

The mass ratio (mass concentration) of tin, manganese, and potassium in the mass ratio represented by tin:manganese:potassium salt of oxide:silica is determined by analyzing the oxidative coupling catalyst using inductively coupled plasma atomic emission spectroscopy (ICP-AES). The mass ratio (mass concentration) of silica can be calculated by subtracting the mass ratio (mass concentration) of each active component (tin, manganese, and potassium) from the total mass of the oxidative coupling catalyst.

The mass ratio represented by tin:manganese:potassium salt of oxide:silica can be adjusted by the concentration of an aqueous solution of each raw material, the amount thereof, and a combination thereof.

The silica which is the carrier of the oxidative coupling catalyst of the present embodiment is not particularly limited, and examples thereof include a granular silica gel, silica powder, and silica obtained by extrusion-molding a composition in which silica powder is dispersed in a binder to mold the composition in a pellet shape (hereinafter, also referred to as “extrusion-molded silica”), and extrusion-molded and sintered silica obtained by sintering the extrusion-molded silica, for example, at 700° C. or higher for 5 hours or longer. Among the examples of silica, from the viewpoint that the particle diameter can be easily adjusted by selecting a die and silica is not broken during sintering at a high temperature even in a case where the particle diameter is large, extrusion-molded silica and extrusion-molded and sintered silica (sintered substance of extrusion-molded silica) are preferable, and extrusion-molded and sintered silica is more preferable.

The specific surface area of the silica is preferably 200 to 500 m²/g, more preferably 250 to 450 m²/g, and still more preferably 300 to 400 m²/g.

The pore volume of the silica is preferably 0.25 to 0.60 cm³/g, more preferably 0.30 to 0.55 cm³/g, and still more preferably 0.40 to 0.50 cm³/g.

In a case where the silica is extrusion-molded and sintered silica, the specific surface area of the extrusion-molded and sintered silica is preferably 80 to 140 m²/g, more preferably 90 to 130 m²/g, and still more preferably 100 to 120 m²/g.

In a case where the silica is extrusion-molded and sintered silica, the pore volume of the extrusion-molded and sintered silica is preferably 0.12 to 0.18 cm³/g, more preferably 0.13 to 0.17 cm³/g, and still more preferably 0.14 to 0.16 cm³/g.

The specific surface area of the oxidative coupling catalyst of the present disclosure is preferably 1.0 to 7.5 m²/g, more preferably 1.3 to 7.0 m²/g, and still more preferably 1.5 to 5.5 m²/g.

The pore volume of the oxidative coupling catalyst of the present disclosure is preferably 0.0010 to 0.0100 cm³/g, more preferably 0.0020 to 0.0080 cm³/g, and still more preferably 0.0030 to 0.0070 cm³/g.

In the present specification, the specific surface area and the pore volume can be measured by a gas adsorption method.

Examples of the binder used for the extrusion-molded silica include a binder containing a methyl cellulose derivative, a polyacrylic acid-based copolymer, a polyurethane-based copolymer, or the like. Among these, a binder containing a methyl cellulose derivative is preferable. Examples of the binder include SELANDER (registered trademark) YB series (manufactured by HighChem Corporation), but the present invention is not limited thereto.

It is preferable that silica as a raw material of the oxidative coupling catalyst of the present embodiment is amorphous. By impregnating amorphous silica with active components such as tin, manganese, potassium, and indium and sintering the silica, the silica exhibits crystallinity and can exhibit a function as a catalyst. Further, the silica as a raw material may be crystalline.

Whether or not the silica is amorphous can be confirmed by X-ray diffraction (XRD). In XRD, in a case where a peak is not observed at a specific diffraction angle, that is, in a case where a diffraction phenomenon is not observed, it is determined that the silica is amorphous.

The methane conversion rate (%) of the oxidative coupling catalyst according to the present embodiment, which is defined by Expression (1), is preferably 13.0% to 25.0%, more preferably 15.0% to 23.0%, and still more preferably 17.9% to 20.0%.

The selectivity (%) of the hydrocarbon having 2 to 4 carbon atoms of the oxidative coupling catalyst according to the present embodiment, which is defined by Expression (2), is preferably 50% to 80%, more preferably 54% to 70%, and still more preferably 55% to 65%.

The yield (%) of the hydrocarbon having 2 to 4 carbon atoms of the oxidative coupling catalyst according to the present embodiment, which is defined by Expression (3), is preferably 7% to 30%, more preferably 9% to 20%, and still more preferably 10% to 15%.

### <<Method for Producing Oxidative Coupling Catalyst>>

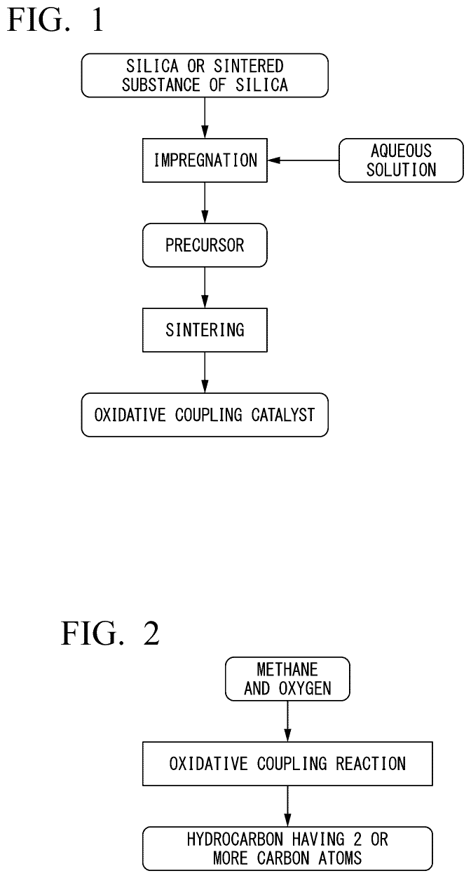

A method for producing the oxidative coupling catalyst of the present disclosure includes a step of bringing an aqueous solution containing an alkali metal salt into contact with silica or a sintered substance of silica to obtain a precursor in which the silica or the sintered substance of silica is impregnated with the aqueous solution, and a step of sintering the precursor at 800° C. or higher to obtain an oxidative coupling catalyst, as shown in FIG. 1.

Hereinafter, each step will be described in more detail.

The aqueous solution containing an alkali metal salt with which silica or a sintered substance of silica is impregnated is obtained by dissolving an alkali metal salt of an oxide containing at least tungsten or zirconium in water. The aqueous solution containing a metal element other than tungsten and zirconium is obtained by dissolving an inorganic compound such as a chloride, a nitrate, a sulfate, a carbonate, a hydrogen carbonate, or an ammonium salt and an organic compound such as an acetate or an oxalate of other metal elements other than tungsten and zirconium in water. In addition, an aqueous solution may be obtained by dissolving a composite oxide such as potassium tungstate in water.

The molar concentration of the metal in the aqueous solution can be appropriately determined according to the performance required for the oxidative coupling catalyst.

In a case where an aqueous solution containing a plurality of kinds of metals is used in the step of obtaining a precursor, an aqueous solution in which each kind of metal is dissolved may be prepared and silica may be sequentially impregnated with each aqueous solution (sequential impregnation method), or silica may be co-impregnated with an aqueous solution in which a plurality of kinds of metals are dissolved (co-impregnation method).

In a case where an aqueous solution containing a plurality of kinds of metals is used, the co-impregnation method is preferable from the viewpoint that the production efficiency of the catalyst is excellent.

In the sequential impregnation method, the content of water in the aqueous solution in which each kind of metal is dissolved is preferably 100 to 500 parts by mass with respect to 100 parts by mass of the compound containing the metal.

In the co-impregnation method, the content of water in the aqueous solution in which a plurality of kinds of metals are dissolved is preferably 100 to 500 parts by mass with respect to 100 parts by mass of the total amount of the compound containing the metals.

In the sequential impregnation method, it is preferable that the silica is impregnated with an aqueous solution containing other metal elements before the silica is impregnated with an aqueous solution containing an alkali metal salt of an oxide containing at least tungsten or zirconium.

In the co-impregnation method, in a case where the aqueous solution in which a plurality of kinds of metals are dissolved contains manganese and a potassium salt of an oxide, the mass ratio represented by manganese:potassium salt of oxide:silica is preferably (0.01 to 5):(0.05 to 10):(85 to 99.9), more preferably (0.5 to 4):(1 to 8):(88 to 98.5), and still more preferably (1 to 3):(2 to 7):(90 to 97). Here, the total mass of the manganese, the potassium salt of an oxide, and the silica is set to 100.

In the co-impregnation method, in a case where an aqueous solution in which a plurality of kinds of metals are dissolved contains tin, manganese, and a potassium salt of an oxide, the mass ratio represented by tin:manganese:potassium salt of oxide:silica is preferably (0.01 to 50):(0.01 to 5):(0.05 to 10):(35 to 99.9), more preferably (2 to 50):(0.5 to 4):(1 to 8):(38 to 96.5), and still more preferably (10 to 20):(1 to 3):(2 to 7):(70 to 87). Here, the total mass of the tin, the manganese, the potassium salt of an oxide, and the silica is set to 100.

In the step of obtaining the sintered substance of the precursor, the temperature (sintering temperature) in a case of sintering the precursor is, for example, 800° C. or higher, preferably 850° C. or higher, and more preferably 850° C. or higher and lower than 900° C. In a case where the sintering temperature is higher than or equal to the above-described lower limits, the alkali metal salt is converted into an oxide containing an alkali metal or a composite oxide thereof, and is supported by a sintered substance of silica as a stable active component. In addition, in a case where the amorphous silica exhibits crystallinity, the catalytic activity is further enhanced. In a case where the sintering temperature is lower than the above-described upper limit, melting and volatilization of the active component of the catalyst can be suppressed, and a decrease in the active component of the catalyst can be suppressed.

In the present specification, the term “sintering temperature” is expressed as a set temperature of an oven or the like in a case of sintering a precursor.

In a case of using the sequential impregnation method, a sintered substance of the precursor can be obtained by impregnating the precursor with an aqueous solution in which a first metal is dissolved, sintering the obtained impregnated substance, impregnating the sintered substance with an aqueous solution in which a second metal is dissolved, sintering the obtained impregnated substance, further impregnating the sintered substance with an aqueous solution in which a third metal is dissolved, and sintering the obtained impregnated substance.

In the sequential impregnation method, it is preferable that the first metal and the second metal are metal elements other than tungsten and zirconium and that the third metal is a metal element containing tungsten and zirconium.

The time (sintering time) for sintering the precursor is, for example, preferably 1 to 24 hours, more preferably 2 to 18 hours, and still more preferably 3 to 12 hours. In a case where the sintering time is longer than or equal to the above-described lower limits, the active component is sufficiently supported by the silica. In addition, from the viewpoint of the productivity, it is preferable that the sintering time is shorter than or equal to the above-described upper limits.

In the present specification, the term “sintering time” is expressed as a time during which heating in an oven or the like is maintained after reaching the sintering temperature.

The method for producing the oxidative coupling catalyst according to the present disclosure may further include a step of extrusion-molding a composition in which silica powder is dispersed in a binder to obtain silica. By using extrusion-molded silica as a carrier of the oxidative coupling catalyst, the particle diameter of the oxidative coupling catalyst can be easily adjusted, and the occurrence of breakage during sintering at a high temperature can be suppressed even in a case where the particle diameter is large.

The shape of the extrusion-molded silica is not particularly limited, and examples thereof include a columnar shape, a ring shape, a spherical shape, a lump shape, and a fibrous shape. The shape of the extrusion-molded silica can be adjusted, for example, by cutting or crushing an extrusion-molded noodle-shaped molded body into any shape.

It is preferable that the method for producing the oxidative coupling catalyst according to the present disclosure further includes a step of sintering a molded body obtained by extrusion-molding the composition at 700° C. or higher to obtain a sintered substance of the silica. By using a sintered body obtained by sintering the extrusion-molded silica (hereinafter, also referred to as “extrusion-molded and sintered silica”) as a carrier, the occurrence of breakage of the silica carrier can be further suppressed in the sintering step of obtaining the oxidative coupling catalyst later. For example, during the sintering, the specific surface area and the pore volume are rapidly reduced (sintered), and thus thermal stress is generated inside the silica. In silica having low toughness, breakage may occur due to inability to withstand the thermal stress. Since the extrusion-molded and sintered silica has toughness that can withstand the thermal stress, the occurrence of breakage of the silica carrier can be further suppressed.

Examples of an index of the mechanical properties of the material related to the breakage include strength indicating static toughness and toughness indicating dynamic toughness. In general, the strength and the toughness are known to establish a trade-off relationship. It is considered that the breakage is greatly affected by the dynamic toughness due to occurrence of the shrinkage deformation accompanied by a decrease in the specific surface area and the pore area during the silica sintering. Therefore, it is preferable that the silica carrier has high toughness, that is, low strength in order to suppress the occurrence of breakage of the silica carrier. The strength can be evaluated by, for example, measuring the strength (crushing strength) of the silica carrier in a case where the silica carrier is crushed.

The crushing strength of the extrusion-molded and sintered silica is, for example, preferably 10 N/mm² or less, more preferably 8 N/mm² or less, and still more preferably 6 N/mm² or less. In a case where the crushing strength of the extrusion-molded and sintered silica is less than or equal to the above-described upper limits, the occurrence of breakage of the silica carrier can be further suppressed. The lower limit value of the crushing strength of the extrusion-molded and sintered silica is not particularly limited, but is, for example, 2 N/mm².

The crushing strength of the extrusion-molded and sintered silica is calculated as, for example, an average value of crushing strengths measured with a Kiya hardness meter for five silica carriers randomly extracted.

The crushing strength of the extrusion-molded and sintered silica can be adjusted by the size, shape, sintering conditions, and the like of the extrusion-molded and sintered silica.

The sintering temperature in a case of obtaining the extrusion-molded and sintered silica (hereinafter, also referred to as “pre-sintering temperature”) is, for example, preferably 400° C. or higher. In a case where the pre-sintering temperature is higher than or equal to the above-described lower limit, the strength can be sufficiently imparted to the silica carrier. The upper limit of the pre-sintering temperature is not particularly limited, and is, for example, 1000° C.

In the present specification, the term “pre-sintering temperature” is expressed as a set temperature of an oven or the like in a case of sintering the extrusion-molded silica.

The sintering time (hereinafter, also referred to as “pre-sintering time”) in a case of obtaining the extrusion-molded and sintered silica is, for example, preferably 1 to 24 hours and more preferably 5 to 10 hours. In a case where the pre-sintering time is longer than or equal to the above-described lower limits, the strength and toughness can be sufficiently imparted to the silica carrier, and the occurrence of breakage of the silica carrier can be further suppressed in the subsequent sintering step of obtaining the oxidative coupling catalyst. In a case where the pre-sintering time is shorter than or equal to the above-described upper limits, the time for obtaining the extrusion-molded and sintered silica can be reduced, and the production efficiency of the oxidative coupling catalyst can be further increased.

In the present specification, the term “pre-sintering time” is expressed as a time during which heating in an oven or the like is maintained after reaching the pre-sintering temperature.

In the method for producing the oxidative coupling catalyst according to the present disclosure, it is preferable that the silica in the step of obtaining silica is amorphous. By impregnating amorphous silica with active components such as tin, manganese, potassium, and indium and sintering the silica, the silica exhibits crystallinity and can exhibit a function as a catalyst. Further, the silica as a raw material may be crystalline.

Examples of the amorphous silica include the above-described granular silica gel, silica powder, extrusion-molded silica, and extrusion-molded and sintered silica.

### <<Method for Producing Hydrocarbons>>

The method for producing hydrocarbons according to the present disclosure is a method for generating hydrocarbons having 2 or more carbon atoms from methane by an oxidative coupling reaction of methane, using the oxidative coupling catalyst according to the present disclosure.

In the method for producing hydrocarbons according to the present disclosure, a hydrocarbon having 2 or more carbon atoms can be produced with a high yield by using the oxidative coupling catalyst according to the present disclosure. In addition, since the oxidative coupling catalyst of the present disclosure is used in the method for producing a hydrocarbon of the present disclosure, a hydrocarbon having 2 or more carbon atoms can be produced with a high yield even at a high temperature under pressure, and the size of the facility that performs the OCM reaction can be reduced.

As shown in FIG. 2, the method for producing hydrocarbons of the present embodiment is a method for generating a hydrocarbon having 2 or more carbon atoms from methane by the oxidative coupling reaction of methane.

Hereinafter, the method for producing hydrocarbons according to the present embodiment will be described in more detail.

The methane used as the reaction raw material may be pure methane or may be a methane-containing gas containing other components in a range where the OCM reaction is not impaired. These methane and methane-containing gas can be obtained from a natural gas, methane-containing gas obtained from a high-temperature coke furnace of coal, methane-containing gas obtained from a hydrogenation reaction of carbon monoxide or carbon dioxide generated from coal decomposition gas or from decomposition of a hydrocarbon derived from a petroleum fraction, and the like. In addition, methane used as a reaction raw material can be obtained by performing isolation or a purification treatment on methane from a methane-containing gas obtained by a fermentation method or the above-described methane-containing gas.

The OCM reaction can be carried out in an atmosphere where oxygen, carbon dioxide, (nitrous) nitrogen oxide, or the like is present, but it is preferable that the OCM reaction is carried out in an atmosphere where oxygen is present.

As an oxygen source provided for the OCM reaction, an oxygen-containing gas such as oxygen, air, and oxygen-enriched air can be used. Further, it is more preferable that the OCM reaction is carried out in an atmosphere where oxygen and steam coexist. This is because a hydroxy radical (OH radical) is generated from steam and active oxygen on the catalyst surface. The OH radical reacts with methane to generate a methyl radical (CH₃ radical). In the oxidative coupling reaction of methane, a reaction mechanism in which ethane and ethylene are generated from a CH₃ radical is considered.

As the steam source, water may be evaporated by a boiler or the like and used, or steam or the like in exhaust gas of a boiler or various chemical plants may be isolated or subjected to a purification treatment as necessary and used. In addition, since steam is also generated even by the OCM reaction, the steam may be used.

The proportion of the oxygen and the steam to the methane provided for the OCM reaction is preferably in a range of 0.05 to 0.2 mol of oxygen and 0 to 0.1 mol of steam with respect to 1 mol of methane. In a case where the proportion of oxygen and steam is in the above-described numerical ranges, a hydrocarbon having 2 or more carbon atoms can be produced with a higher yield and more safely.

In the OCM reaction, an inert gas such as nitrogen, helium, or argon may be present in the reaction atmosphere.

The temperature of the OCM reaction (reaction temperature) is, for example, preferably 500° C. to 1,100° C., more preferably 600° C. to 1,000° C., and still more preferably 700° C. to 900° C. In a case where the reaction temperature is higher than or equal to the above-described lower limits, a practical reaction rate can be obtained. In a case where the reaction temperature is lower than or equal to the above-described upper limits, side reactions such as a steam reforming reaction, a combustion reaction, and a polymerization reaction can be suppressed, and the yield of the hydrocarbon having 2 or more carbon atoms can be further increased. In addition, the durability of the catalyst can be further improved by suppressing melting and volatilization of the active component of the catalyst.

In the present specification, the term “reaction temperature” is expressed as a temperature inside a catalyst layer of a reactor or the like in the OCM reaction.

It is preferable that the OCM reaction is carried out under a pressurized condition. By carrying out the OCM reaction under a pressurized condition, the gas volume can be reduced, and the size of the reactor that performs the OCM reaction can be reduced. In addition, the power of a compressor or the like that supplies a process gas to a purification step and a separation step on a downstream of the reactor can be reduced.

The pressure (reaction pressure) in the OCM reaction is, for example, preferably 0.4 to 1.2 MPa, more preferably 0.5 to 1.1 MPa, and still more preferably 0.6 to 1.0 MPa. In a case where the reaction pressure is greater than or equal to the above-described lower limits, the size of the reactor that performs the OCM reaction can be further reduced. In a case where the reaction pressure is less than or equal to the above-described upper limits, side reactions such as a combustion reaction and a polymerization reaction can be suppressed, and a decrease in the yield of the hydrocarbon having 2 or more carbon atoms can be suppressed.

In the present specification, the term “reaction pressure” is expressed as the total pressure of the raw material gas provided for the reaction. In the present specification, the pressure is measured by a pressure gauge equipped in the reaction container.

Examples of the facility that performs the OCM reaction include a fixed bed reactor that circulates a mixture of methane or a methane-containing gas and oxygen or an oxygen-containing gas and preferably an atmospheric gas containing steam with the raw material through a reactor filled with a catalyst. The facility that performs the OCM reaction may be a fluidized bed reactor or a moving bed reactor. In addition, a reactor that enables the use of a membrane reactor or a divided feed method of injecting oxygen by performing division for individual catalyst layers may be used for the purpose of suppressing the combustion of raw material methane due to a high oxygen concentration in a catalyst layer inlet portion.

The space velocity in the OCM reaction is, for example, preferably 1,000 to 5,000,000/h and more preferably 10,000 to 500,000/h. In a case where the space velocity in the OCM reaction is in the above-described numerical ranges, the yield of the hydrocarbon having 2 or more carbon atoms can be further increased.

Here, the term “space velocity” is also referred to as GHSV, and denotes a total volume flow rate per unit volume of a catalyst layer per hour (0° C., 1 atm conditions) of a raw material gas. The volume of the catalyst layer denotes a volume including the catalyst filling a reaction tube and the voids thereof.

The reactor may be filled with one kind of catalyst, or filled with a mixture of a plurality of kinds of catalysts having different activities or with the catalysts in individual layers. As desired, the reactor can be filled with a catalyst such that the activity changes from the inlet to the outlet of the reactor, using a plurality of kinds of catalysts having different activities or an inert inorganic substance used as one kind of catalyst and a diluent.

The composition of the reactor outlet gas, that is, the gas containing hydrocarbons having 2 or more carbon atoms generated by the OCM reaction varies depending on the reaction raw materials. The hydrocarbons having 2 or more carbon atoms, which are the target products in the outlet gas, are introduced into known separation and purification facilities, and each of the components is recovered, purified, recycled, and discharged, whereby the required target products, for example, ethylene, ethane, propane, propylene, and butane can be obtained.

Hereinbefore, the embodiments of the present disclosure have been described in detail, each configuration in each embodiment and combinations thereof are merely examples, and addition, omission, substitution, and other changes of the configuration can be made without departing from the scope of the present disclosure. In addition, the present disclosure is not limited to the embodiments, and is limited only by the scope of the claims.

### EXAMPLES

Hereinafter, the present disclosure will be described in more detail with reference to examples, but the present disclosure is not limited to these examples.

The raw materials used in the present examples are as follows.

(Raw materials used)
Mn(NO₃)₂·6H₂O:manganese (II) nitrate hexahydrate
SnCl₄·5H₂O:tin (IV) chloride pentahydrate
K₂WO₄:potassium tungstate
SiO₂:silica powder
In(NO₃)₂·3H₂O:indium (III) nitrate trihydrate

### <<Preparation of Extrusion-Molded Silica>>

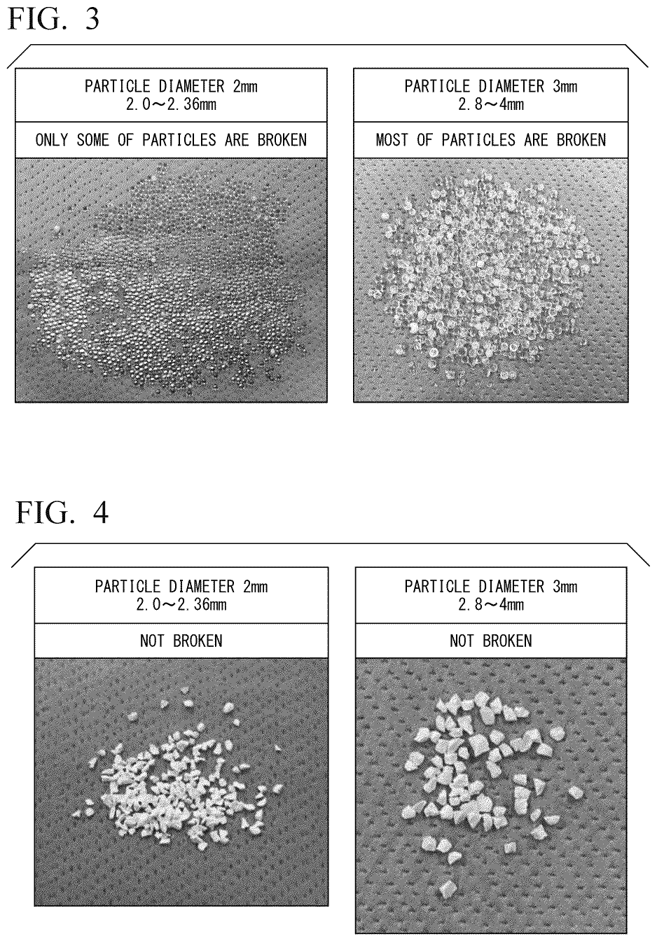

In a case where silica gel particles selected as a material of a catalyst carrier in the related art are sintered at 900° C. or higher, the specific surface area (pore volume) is significantly reduced, a stress is applied to the particles, and the particles are broken in a case where the average particle diameter is 3 mm or greater (see FIG. 3).

Therefore, an experiment of sintering particles at 900° C. for 8 hours was performed using silica extruded and molded into a pellet shape, and crushed to have a uniform particle diameter (hereinafter, also referred to as “extrusion-molded silica”). The results thereof are shown in FIG. 4.

As shown in FIG. 4, the extrusion-molded silica did not break even in a case of having an average particle diameter of 3 mm. This is because the extrusion-molded silica has high resistance (toughness) to a stress.

### <<Measurement of Specific Surface Area and Pore Area>>

Freshly extrusion-molded silica before sintering, extrusion-molded silica (extrusion-molded and sintered silica) after sintering at 900° C. for 8 hours, and an oxidative coupling catalyst in which an alkali metal salt was supported on the extrusion-molded and sintered silica were prepared, and the specific surface area and the pore area of each of the prepared materials were measured. The results thereof are listed in Table 1.

TABLE 1

| | Mass of | Specific surface | | Pore volume | |
|---|---|---|---|---|---|
| | sample | area (m²/g) | | (cm³/g) | |
| | (g) | n = 2 | Average | n = 2 | Average |
| Extrusion-molded | 0.2614 | 327.84 | 326 | 0.4611 | 0.46 |
| silica | 0.2585 | 324.76 | | 0.4571 | |
| Extrusion-molded | 0.2651 | 115.58 | 116 | 0.1510 | 0.15 |
| and sintered silica | 0.2597 | 116.22 | | 0.1523 | |
| Oxidative coupling | 0.2644 | 2.54 | 2.5 | 0.0052 | 0.005 |
| catalyst | 0.2647 | 2.44 | | 0.0050 | |

As listed in Table 1, it was confirmed that the specific surface area and the pore area were reduced by sintering the extrusion-molded silica. Further, it was confirmed that the specific surface area and the pore area were significantly reduced by allowing the extrusion-molded silica after sintering to support the alkali metal salt. It is considered that sintering occurred due to high-temperature sintering.

### <<X-Ray Diffraction (XRD)>>

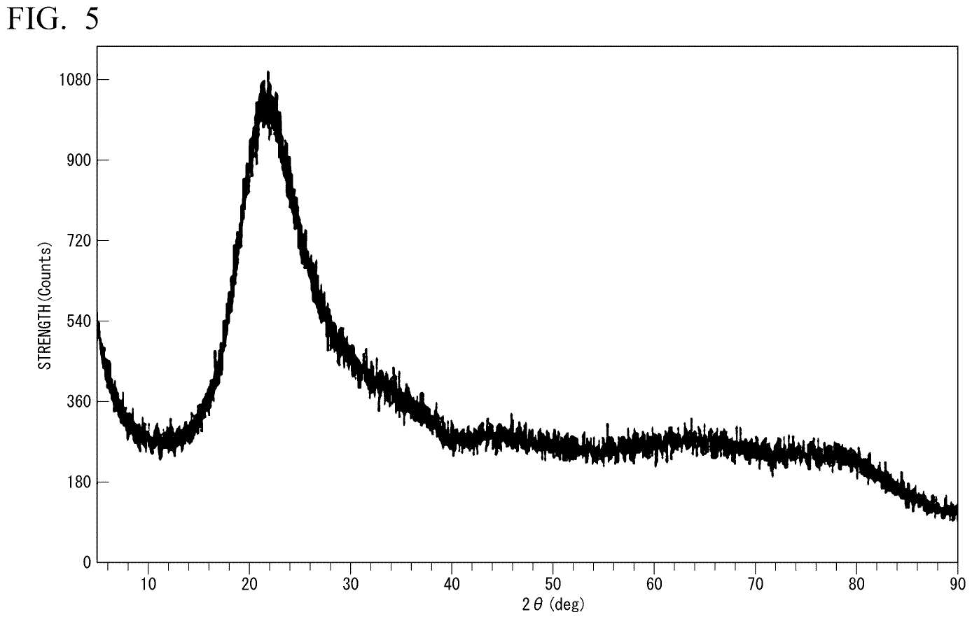

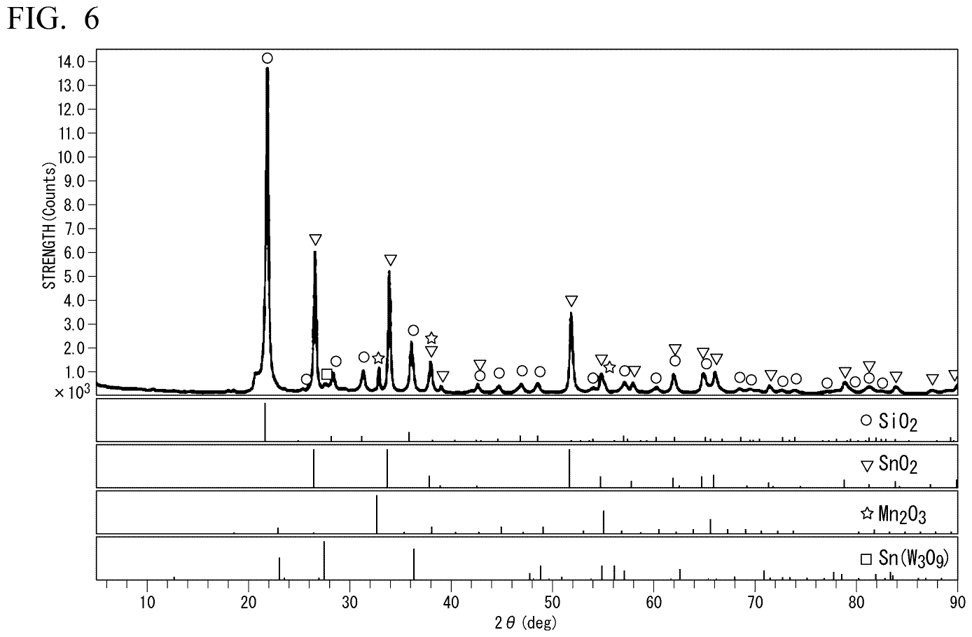

X-ray diffraction (XRD) of the above-described extrusion-molded and sintered silica and an oxidative coupling catalyst in which an alkali metal salt was supported on the extrusion-molded and sintered silica was performed. The results thereof are shown in FIGS. 5 and 6.

As shown in FIG. 5, it can be confirmed that the extrusion-molded and sintered silica has an amorphous structure.

On the contrary, as shown in FIG. 6, in the oxidative coupling catalyst in which the alkali metal salt was supported on the extrusion-molded silica after sintering, a diffraction phenomenon was observed, and the silica was confirmed to be crystallized.

As described above, it was confirmed that the oxidative coupling catalyst of the present disclosure does not break even in a case of being crystallized.

### <<Measurement of Crushing Strength>>

A columnar extrusion-molded and sintered silica having a diameter of 5 mm and a height of 5 mm and a spherical silica gel having a diameter of 4 mm were prepared, and a compressive force (N) during crushing was measured using a Kiya hardness tester (043019-D, manufactured by FUJIWARA SCIENTIFIC CO., LTD.). The crushing strength (N/mm²) was determined by dividing the compressive force by the area of the silica carrier ((5/2)²×π(mm²) in a case of the extrusion-molded and sintered silica, and (4/2)²×π(mm²) in a case of the silica gel). The results of measuring the crushing strength in a case of n=5 are listed in Table 2.

TABLE 2

| | | n = 1 | n = 2 | n = 3 | n = 4 | n = 5 | Average |
|---|---|---|---|---|---|---|---|
| Extrusion- | Compressive | 119.6 | 93.1 | 89.2 | 88.2 | 147.0 | 107.4 |
| molded | force (N) | | | | | | |
| silica | Crushing | 6.1 | 4.7 | 4.5 | 4.5 | 7.5 | 5.5 |
| φ 5 mm × | strength | | | | | | |
| 5 mm | (N/mm²) | | | | | | |
| Spherical | Compressive | 215.6 | 289.1 | 183.3 | 294.0 | 213.6 | 239.1 |
| silica gel | force (N) | | | | | | |
| Φ 4 mm | Crushing | 17.2 | 23.0 | 14.6 | 23.4 | 17.0 | 19.0 |
| | strength | | | | | | |
| | (N/mm²) | | | | | | |

As listed in Table 2, the crushing strength of the silica gel was 2 times or greater the crushing strength of the extrusion-molded and sintered silica. As shown in this result, it was found that the extrusion-molded and sintered silica has sufficiently high toughness and a high possibility of not being broken during sintering.

### <<Production of Oxidative Coupling Catalyst>>

### Example 1

6.27 mL of ultrapure water, which was the amount three times the amount of 2.09 g of a raw material powder of Mn(NO₃)²·6H₂O, was added to the raw material powder, and the mixture was stirred to prepare a Mn raw material aqueous solution. 54.6 mL of ultrapure water, which was the amount three times the amount of 18.2 g of SiO₂ powder, was added to the SiO₂ powder, the Mn raw material aqueous solution was added to the SiO₂ powder, and the SiO₂ powder was impregnated with the Mn raw material aqueous solution, thereby obtaining an impregnated substance I. The impregnated substance I was heated and dried at 130° C. for 5 hours in an air atmosphere to evaporate the moisture. The impregnated substance I after being dried was heated at a temperature increasing rate of 2° C./min, sintered at 900° C. for 8 hours, and cooled at a cooling rate of 20° C./min, thereby obtaining a sintered substance (sintered substance I) of the impregnated substance I.

Next, 3.54 mL of ultrapure water, which was the amount three times the amount of 1.18 g of the raw material powder of SnCl₄·5H₂O, was added to the raw material powder, and the mixture was stirred to prepare a Sn raw material aqueous solution. Ultrapure water was added to the sintered substance I in an amount three times the weight of the sintered substance I, and the Sn raw material aqueous solution was added thereto for impregnation, thereby obtaining an impregnated substance II. The impregnated substance II was heated and dried at 130° C. for 5 hours in an air atmosphere to evaporate the moisture. The impregnated substance II after being dried was heated at a temperature increasing rate of 2° C./min, sintered at 900° C. for 8 hours, and cooled at a cooling rate of 20° C./min, thereby obtaining a sintered substance (sintered substance II) of the impregnated substance II.

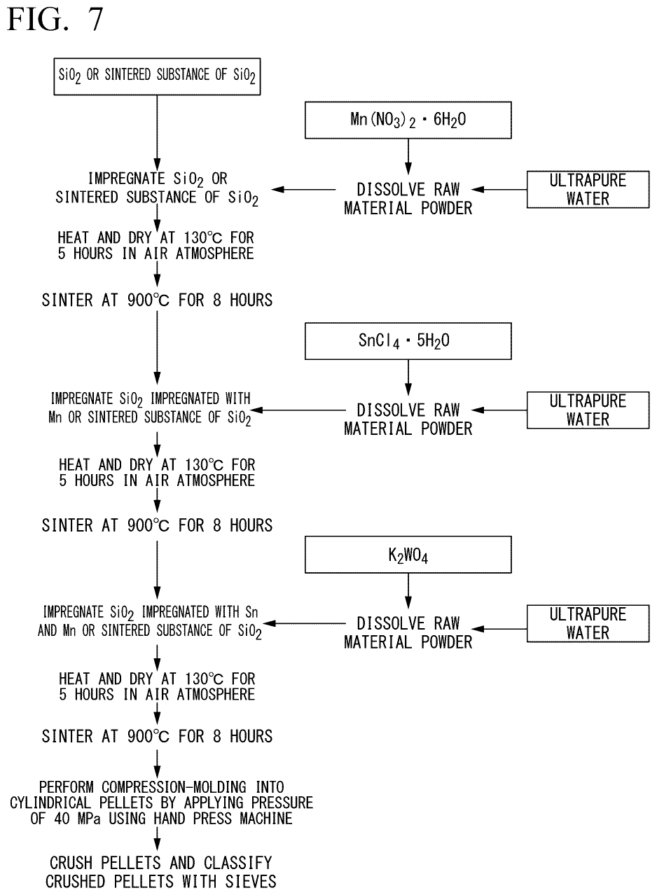

Next, 3 mL of ultrapure water, which was the amount three times the amount of 1.00 g of the raw material powder of K₂WO₄, was added to the raw material powder, and the mixture was stirred to prepare a K raw material aqueous solution. Ultrapure water was added to the sintered substance II in an amount three times the weight of the sintered substance II, and the K raw material aqueous solution was added thereto for impregnation, thereby obtaining an impregnated substance III (precursor). The impregnated substance III was heated and dried at 130° C. for 5 hours in an air atmosphere to evaporate the moisture. The impregnated substance III after being dried was heated at a temperature increasing rate of 2° C./min, sintered at 900° C. for 8 hours, and cooled at a cooling rate of 20° C./min, thereby obtaining a sintered substance (sintered substance III) of the impregnated substance III. A hand press machine having a diameter of 20 mm was filled with the obtained sintered substance III, and the sintered substance III was compression-molded into a cylindrical pellet by applying a pressure of 40 MPa. The obtained pellets were crushed with a crusher and classified with sieves having opening diameters of 250 μm and 500 μm to obtain an oxidative coupling catalyst having a particle diameter of 250 to 500 μm (sequential impregnation method, see FIG. 7).

### Example 2

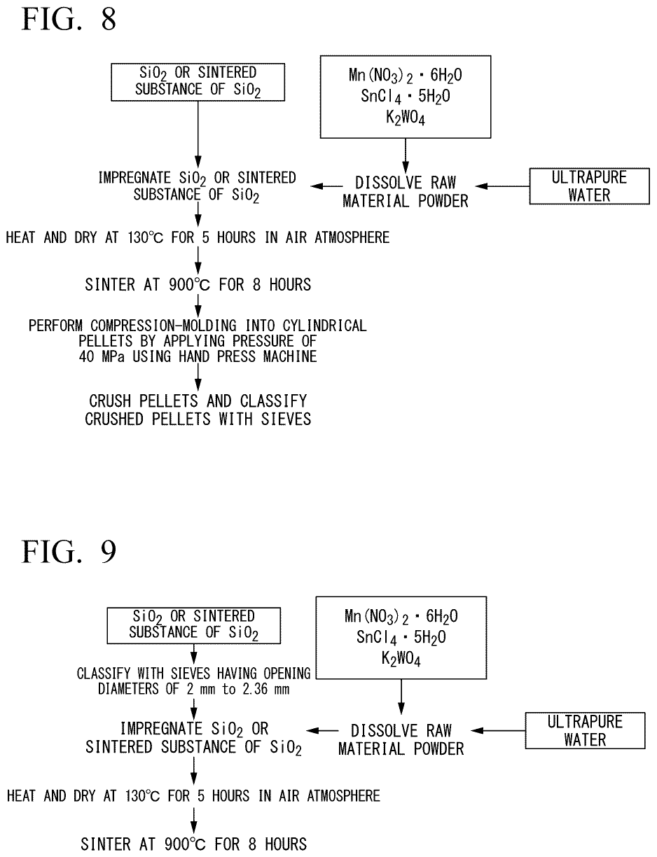

12.8 mL of ultrapure water, which was the amount three times the amount of 2.09 g of raw material powder of Mn(NO₃)₂·6H₂O, 1.18 g of raw material powder of SnCl₄·5H₂O, and 1.00 g of raw material powder of K₂WO₄, was added to the raw material powders, and the mixture was stirred to prepare a raw material aqueous solution. The raw material aqueous solution was added to 14.6 g of SiO₂ powder, 54.6 mL of ultrapure water was added thereto, the raw material aqueous solution was brought into contact with SiO₂ powder, and the SiO₂ powder was impregnated with the raw material aqueous solution, thereby obtaining a precursor. The obtained precursor was heated and dried at 130° C. for 5 hours in an air atmosphere to evaporate the moisture. The dried precursor was heated at a temperature increasing rate of 2° C./min, sintered at 900° C. for 8 hours, and cooled at a cooling rate of 20° C./min, thereby obtaining a sintered substance (sintered substance IV) of the precursor. A hand press machine having a diameter of 20 mm was filled with the obtained sintered substance IV, and the sintered substance IV was compression-molded into a cylindrical pellet by applying a pressure of 40 MPa. The obtained pellets were crushed with a crusher and classified with sieves having opening diameters of 250 μm and 500 μm to obtain an oxidative coupling catalyst having a particle diameter of 250 to 500 μm (co-impregnation method, see FIG. 8).

### Example 3

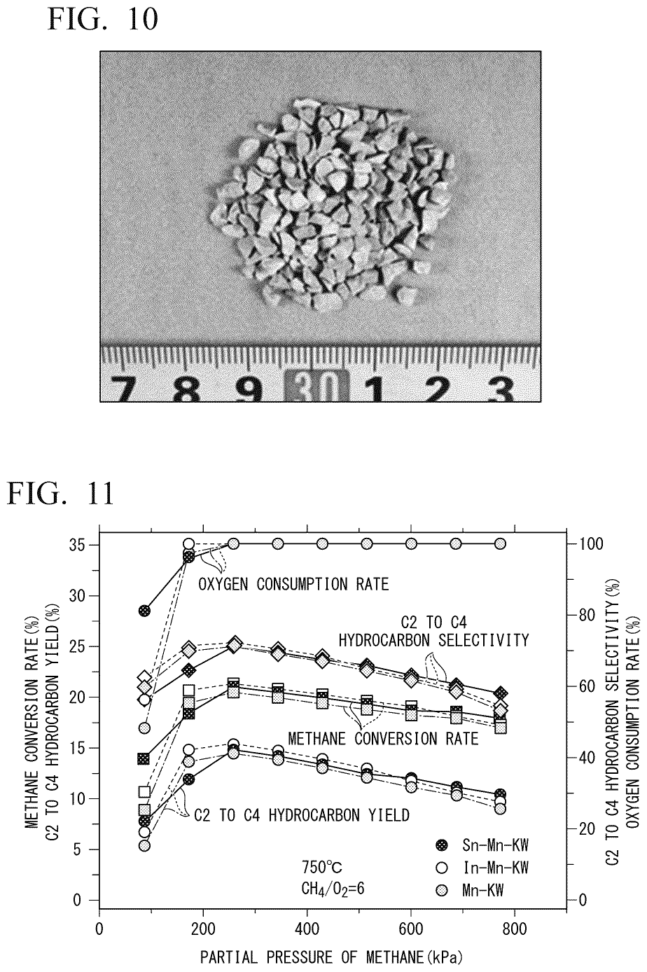

Granular silica or extrusion-molded and sintered silica was prepared and classified with sieves having opening diameters of 2 mm and 2.36 mm to obtain a silica carrier having a particle diameter of 2 to 2.36 mm. The raw material aqueous solution prepared in the same manner as in Example 2 was added to 14.6 g of the obtained silica carrier, 54.6 mL of ultrapure water was added thereto, the raw material aqueous solution was brought into contact with the silica carrier, and the silica carrier was impregnated with the raw material aqueous solution, thereby obtaining a precursor. The obtained precursor was heated and dried at 130° C. for 5 hours in an air atmosphere to evaporate the moisture. The dried precursor was heated at a temperature increasing rate of 2° C./min, sintered at 900° C. for 8 hours, and cooled at a cooling rate of 20° C./min to obtain a granular oxidative coupling catalyst as shown in FIG. 10 (co-impregnation method, see FIG. 9).

### Example 4

An oxidative coupling catalyst having a particle diameter of 250 to 500 μm was obtained by the same method as in Example 2 except that 1.237 g of a raw material powder of In(NO₃)₂·3H₂O was used instead of 1.18 g of the raw material powder of SnCl₄·5H₂O.

### Example 5

An oxidative coupling catalyst having a particle diameter of 250 to 500 μm was obtained by the same method as in Example 2 except that the raw material powder of SnCl₄·5H₂O was not added.

### <<Production of Hydrocarbons>>

The following three kinds of oxidative coupling catalysts produced in Examples 2, 4, and 5 were prepared. In the following formulae, “wt %” represents the proportion of the mass of a metal element with respect to the total mass of an oxidative coupling catalyst.
Sn—Mn—KW:Sn (2 wt %) Mn (2 wt %) K₂WO₄ (5 wt %)/SiO₂
In—Mn—KW: In(2 wt %) Mn (2 wt %) K₂WO₄ (5 wt %)/SiO₂
Mn—KW:Mn (2 wt %) K₂WO₄ (5 wt %)/SiO₂

A pressurizable reactor was filled with the above-described three kinds of oxidative coupling catalysts, and a hydrocarbon was produced by an oxidative coupling reaction of methane using a natural gas containing methane as a raw material. In this case, an inert quartz tube having no catalytic action was used as the reactor.

Here, the yield of the hydrocarbon having 2 to 4 carbon atoms in the generated gas, the methane conversion rate, the selectivity of the hydrocarbon having 2 to 4 carbon atoms, and the oxygen consumption rate were measured with respect to the partial pressure of methane in the raw material gas. The results thereof are shown in FIG. 11.

Here, the methane conversion rate is defined by Expression (1).

$$M ethane conversion rate ( \% ) = ( reactor inlet methane flow rate - reactor outlet methane flow rate ) / reactor inlet methane flow rate \times 100 \qquad ( 1 )$$

The selectivity of the hydrocarbon having 2 to 4 carbon atoms (C2 to C4 hydrocarbon selectivity) is defined by Expression (2).

$$C 2 to C 4 hydrocarbon selectivity ( \% ) = ( C 2 to C 4 hydrocarbon flow rate in terms of methane ) ( reactor inlet methane flow rate - reactor outlet methane flow rate ) \times 100 \qquad ( 2 )$$

In addition, the yield of the hydrocarbon having 2 to 4 carbon atoms (C2 to C4 hydrocarbon yield) is defined by Expression (3).

$$C 2 to C 4 hydrocarbon yield ( \% ) = ( methane conversion rate ) \times ( C 2 to C 4 hydrocarbon selectivity ) \times 100 \qquad ( 3 )$$

Further, each flow rate in the expressions is in units of the molar flow rate (for example, kgmol/h), and “C2 to C4 hydrocarbon flow rate in terms of methane” in Expression (2) denotes the flow rate in terms of methane obtained by multiplying the flow rate of each compound of hydrocarbons having 2 to 4 carbon atoms, such as ethane, ethylene, propane, and propylene, by the number of carbon atoms thereof.

As shown in FIG. 11, it was confirmed that in a case where “Sn—Mn—KW” was used as the oxidative coupling catalyst, the partial pressure of methane was 500 kPa or greater, and the selectivity of the hydrocarbon having 2 to 4 carbon atoms was improved as compared with a case where other oxidative coupling catalysts were used. This means that, in a case where tin is added to the oxidative coupling catalyst, the selectivity of the hydrocarbon having 2 to 4 carbon atoms, which is a target product, is improved under a pressurized condition (for example, 500 kPa or greater).

Therefore, in a case where “Sn—Mn—KW” is used as the oxidative coupling catalyst, a high selectivity can be obtained even at a high temperature under pressure, and thus it is expected that this leads to the size reduction of the facility that performs the OCM reaction, and the reduction of the power of a compressor or the like that supplies a process gas to a purification step and a separation step on a downstream of the reactor.

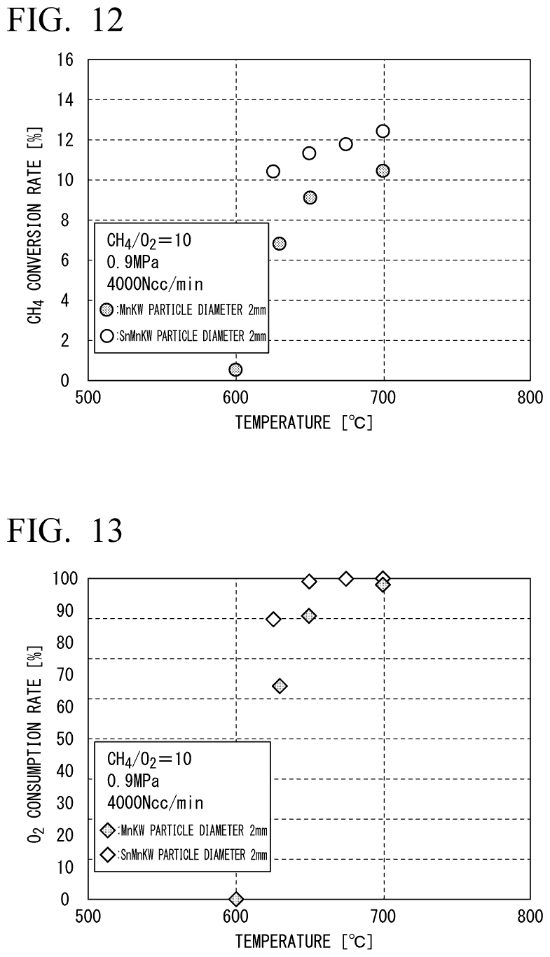

In a case where an oxidative coupling reaction of methane was carried out using an oxidative coupling catalyst (Sn (20 wt %) Mn (2 wt %) K₂WO₄ (5 wt %)/SiO₂) supporting “Sn—Mn—KW” prepared by changing the concentration of the active component using the method in Example 3, and in a case where an oxidative coupling reaction of methane was carried out using silica gel particles supporting “Mn—KW”, the conversion rate of methane and the oxygen consumption rate were measured. The performance was evaluated under the conditions of a total pressure of 0.9 MPa, a gas supply temperature to the catalyst layer of 600° C. to 700° C., a molar ratio of 10 (mol/mol) between methane and oxygen (CH₄/O₂), a flow rate of the raw material gas of 4,000 Ncc/min, and an amount of catalyst of 2.3 g. The results thereof are shown in FIGS. 12 and 13.

As shown in FIG. 12, it was confirmed that the conversion rate of methane was improved in a case where “Sn—Mn—KW” is supported on the silica carrier.

As shown in FIG. 13, it was confirmed that in a case where “Sn—Mn—KW” is supported on the silica carrier, the oxygen consumption rate of 100% was maintained even at 650° C., and high catalytic activity was maintained even at a relatively low temperature (for example, about 650° C.).

According to the methods of Examples 1 and 2 described above, oxidative coupling catalysts in which the composition of the active component was changed were prepared (Composition Examples 1 to 10). The BET specific surface area of the obtained oxidative coupling catalyst was measured. In addition, a pressurizable reactor was filled with the above-described 9 kinds of oxidative coupling catalysts (Composition Examples 1 and 2 and Composition Examples 4 to 10), and a hydrocarbon was produced by an oxidative coupling reaction of methane using a natural gas containing methane as a raw material. In this case, an inert quartz tube was used as the reactor.

Here, the yield of the hydrocarbon having 2 to 4 carbon atoms in the generated gas, the methane conversion rate, and the selectivity of the hydrocarbon having 2 to 4 carbon atoms were measured with respect to the partial pressure of methane in the raw material gas. The performance was evaluated under the conditions of a total pressure of 0.9 MPa, a gas supply temperature to the catalyst layer of 750° C., a molar ratio (CH₄/O₂) of 6 (mol/mol) between methane and oxygen, a flow rate of the raw material gas of 240 Ncc/min, and an amount of catalyst of 200 mg. The results thereof are listed in Table 3.

TABLE 3

| | | | | | | | Methane | | |
|---|---|---|---|---|---|---|---|---|---|
| | Active component | | | | | BET specific | conversion | C₂₊ | C₂₊ |
| | (% by mass) | | | | | surface area | rate | selectivity | yield |
| | In | Sn | Mn | K₂WO₄ | Reagent | m²/g | % | % | % |
| Composition | — | — | 2 | 5 | K₂WO₄ | 2.4 | 17.6 | 53.4 | 9.4 |
| Example 1 | | | | | Mn(NO₃)₂•6H₂O | | | | |
| | | | | | SiO₂ | | | | |
| Composition | 2 | — | 2 | 5 | K₂WO₄ | 2.7 | 17.6 | 53.3 | 9.4 |
| Example 2 | | | | | Mn(NO₃)₂•6H₂O | | | | |
| | | | | | In(NO₃)₃•3H₂O | | | | |
| Composition | — | 10 | — | 5 | K₂WO₄ | 5.2 | — | — | — |
| Example 3 | | | | | Mn(NO₃)₂•6H₂O | | | | |
| Composition | — | 20 | — | 5 | SnCl₄•5H₂O | 7.2 | 13.7 | 54.0 | 7.4 |
| Example 4 | | | | | | | | | |
| Composition | — | 2 | 2 | 5 | | 3.5 | 18.0 | 56.2 | 10.1 |
| Example 5 | | | | | | | | | |
| Composition | — | 10 | 2 | 5 | | 3.7 | 18.1 | 59.9 | 10.9 |
| Example 6 | | | | | | | | | |
| Composition | — | 20 | 2 | 5 | | 4.0 | 18.7 | 59.9 | 11.2 |
| Example 7 | | | | | | | | | |
| Composition | — | 50 | 2 | 5 | | 3.8 | 18.5 | 60.8 | 11.2 |
| Example 8 | | | | | | | | | |
| Composition | — | 20 | 2 | 2.5 | | 5.2 | 18.2 | 56.4 | 10.3 |
| Example 9 | | | | | | | | | |
| Composition | — | 20 | 2 | 10 | | 1.8 | 18.1 | 61.1 | 11.1 |
| Example 10 | | | | | | | | | |

As listed in Table 3, in a case of using any oxidative coupling catalyst, a high value of 50% or greater was obtained as the selectivity of the hydrocarbon having 2 to 4 carbon atoms. In addition, the yield of the hydrocarbon having 2 to 4 carbon atoms was a satisfactory value of 7% or greater even in a case of using any oxidative coupling catalyst.

As shown in the results described above, according to the oxidative coupling catalyst of the present disclosure, it was confirmed that a hydrocarbon having 2 or more carbon atoms can be produced with a high yield even at a high temperature under pressure, and the size of the facility that performs the OCM reaction can be reduced.

### Appendix

The oxidative coupling catalyst, the method for producing an oxidative coupling catalyst, and the method for producing hydrocarbons in the above-described embodiments are, for example, understood as follows.
(1) The oxidative coupling catalyst according to a first aspect is an oxidative coupling catalyst that generates a hydrocarbon having 2 or more carbon atoms from methane by an oxidative coupling reaction of methane, and is a sintered substance of silica on which an alkali metal salt is supported.

According to the above-described configuration, even in a case where the OCM reaction is carried out at a high temperature under pressure, the carrier of the oxidative coupling catalyst can be suppressed from being broken. Therefore, a hydrocarbon having 2 or more carbon atoms can be produced with a high yield even at a high temperature under pressure, and the size of the facility that performs the OCM reaction can be reduced.
(2) The oxidative coupling catalyst according to a second aspect is the oxidative coupling catalyst according to (1), in which the sintered substance of silica on which the alkali metal salt is supported exhibits crystallinity.

According to the above-described configuration, the catalytic activity can be further enhanced.
(3) The oxidative coupling catalyst according to a third aspect is the oxidative coupling catalyst according to (1) or (2), in which the alkali metal salt is an alkali metal salt of an oxide containing at least one of tungsten or zirconium.

According to the above-described configuration, the yield of the hydrocarbon having 2 to 4 carbon atoms in the OCM reaction can be further increased.
(4) The oxidative coupling catalyst according to a fourth aspect is the oxidative coupling catalyst according to (3), in which the alkali metal salt is a potassium salt of the oxide.

According to the above-described configuration, the catalytic activity is high, and the heat resistance is more excellent.
(5) The oxidative coupling catalyst according to a fifth aspect is the oxidative coupling catalyst according to (4), in which manganese is further supported.

According to the above-described configuration, the yield of the hydrocarbon having 2 to 4 carbon atoms in the OCM reaction can be further increased.
(6) The oxidative coupling catalyst according to a sixth aspect is the oxidative coupling catalyst according to (5), in which a mass ratio represented by manganese:potassium salt of oxide:silica is (0.01 to 5):(0.05 to 10):(85 to 99.9).

According to the above-described configuration, in the OCM reaction, the selectivity of the hydrocarbon having 2 to 4 carbon atoms can be further increased even under a pressurized condition.
(7) The oxidative coupling catalyst according to a seventh aspect is the oxidative coupling catalyst according to (5), in which tin is further supported.

According to the above-described configuration, the selectivity of the hydrocarbon having 2 to 4 carbon atoms can be further increased even under a pressurized condition.
(8) The oxidative coupling catalyst according to an eighth aspect is the oxidative coupling catalyst according to (7), in which a mass ratio represented by tin:manganese:potassium salt of oxide:silica is (0.01 to 50):(0.01 to 5):(0.05 to 10):(35 to 99.9).

According to the above-described configuration, in the OCM reaction, the selectivity of the hydrocarbon having 2 to 4 carbon atoms can be further increased even under a pressurized condition.
(9) The oxidative coupling catalyst according to a ninth aspect is the oxidative coupling catalyst according to any one of (1) to (8), in which the oxidative coupling catalyst has an average particle diameter of 1 to 5 mm.

According to the above-described configuration, the yield of the hydrocarbon having 2 to 4 carbon atoms in the OCM reaction can be further increased. In addition, the pressure loss in a case where the OCM reaction is carried out under a pressurized condition can be further reduced, and the size of the facility that performs the OCM reaction can be reduced.
(10) A method for producing an oxidative coupling catalyst according to a tenth aspect is a method for producing an oxidative coupling catalyst that generates a hydrocarbon having 2 or more carbon atoms from methane by an oxidative coupling reaction of methane, the method including: a step of bringing an aqueous solution containing an alkali metal salt into contact with silica or a sintered substance of silica to obtain a precursor in which the silica or the sintered substance of silica is impregnated with the aqueous solution; and a step of sintering the precursor at 800° C. or higher to obtain the oxidative coupling catalyst.

According to the above-described configuration, a sintered substance of silica on which an alkali metal salt is supported is obtained. Therefore, even in a case where the OCM reaction is carried out at a high temperature under pressure, the carrier of the oxidative coupling catalyst can be suppressed from being broken. As a result, a hydrocarbon having 2 or more carbon atoms can be produced with a high yield even at a high temperature under pressure, and the size of the facility that performs the OCM reaction can be reduced.
(11) The method for producing an oxidative coupling catalyst according to an eleventh aspect is the method for producing an oxidative coupling catalyst according to (10), the method further including: a step of extrusion-molding a composition in which silica powder is dispersed in a binder to obtain the silica.

According to the above-described configuration, the particle diameter of the oxidative coupling catalyst is easily adjusted, and the heat resistance is more excellent.
(12) The method for producing an oxidative coupling catalyst according to a twelfth aspect is the method for producing an oxidative coupling catalyst according to (11), the method further including: a step of sintering a molded body obtained by extrusion-molding the composition at 700° C. or higher to obtain the sintered substance of silica.

According to the above-described configuration, in the step of obtaining a sintered substance of a precursor later, the occurrence of breakage of the sintered substance can be further suppressed.
(13) The method for producing an oxidative coupling catalyst according to a thirteenth aspect is the method for producing an oxidative coupling catalyst according to (12), the method further including: a step of obtaining the sintered substance of silica, in which the sintered substance of silica has a crushing strength of 10 N/mm² or less.

According to the above-described configuration, in the step of obtaining a sintered substance of a precursor later, the occurrence of breakage of the sintered substance can be further suppressed.
(14) The method for producing an oxidative coupling catalyst according to a fourteenth aspect is the method for producing an oxidative coupling catalyst according to any one of (10) to (13), in which the silica is amorphous.

According to the above-described configuration, in the step of obtaining a sintered substance of a precursor later, the occurrence of breakage of the sintered substance can be further suppressed.
(15) A method for producing hydrocarbons according to a fifteenth aspect is a method of generating a hydrocarbon having 2 or more carbon atoms from methane by an oxidative coupling reaction of methane using the oxidative coupling catalyst according to any one of (1) to (9).

According to the above-described configuration, a hydrocarbon having 2 or more carbon atoms can be produced with a high yield. In addition, a hydrocarbon having 2 or more carbon atoms can be produced with a high yield even at a high temperature under pressure, and the size of the facility that performs the OCM reaction can be reduced.
(16) The method for producing hydrocarbons according to a sixteenth aspect is the method for producing hydrocarbons according to (15), in which the oxidative coupling reaction is performed under a pressurized condition.

According to the above-described configuration, the size of the facility that performs the OCM reaction can be reduced. As a result, the power required for the facility that performs the OCM reaction can be reduced.
(17) The method for producing hydrocarbons according to a seventeenth aspect is the method for producing hydrocarbons according to (15) or (16), in which the pressure under a pressurized condition is 0.9 MPa or greater.

According to the above-described configuration, the size of the facility that performs the OCM reaction can be reduced.
(18) The method for producing hydrocarbons according to an eighteenth aspect is the method for producing hydrocarbons according to any one of (15) to (17), in which the oxidative coupling reaction is performed at 500° C. or higher.

According to the above-described configuration, a decrease in catalytic activity can be suppressed.
(19) Use of a sintered substance of silica on which an alkali metal salt is supported, for producing an oxidative coupling catalyst according to a nineteenth aspect, in which the oxidative coupling catalyst is an oxidative coupling catalyst that generates a hydrocarbon having 2 or more carbon atoms from methane by an oxidative coupling reaction of methane.

According to the above-described configuration, a hydrocarbon having 2 or more carbon atoms can be produced with a high yield. In addition, a hydrocarbon having 2 or more carbon atoms can be produced with a high yield even at a high temperature under pressure, and the size of the facility that performs the OCM reaction can be reduced.
(20) The use of a sintered substance of silica on which an alkali metal salt is supported, as an oxidative coupling catalyst according to a twentieth aspect, in which the oxidative coupling catalyst is an oxidative coupling catalyst that generates a hydrocarbon having 2 or more carbon atoms from methane by an oxidative coupling reaction of methane.

According to the above-described configuration, a hydrocarbon having 2 or more carbon atoms can be produced with a high yield. In addition, a hydrocarbon having 2 or more carbon atoms can be produced with a high yield even at a high temperature under pressure, and the size of the facility that performs the OCM reaction can be reduced.

### INDUSTRIAL APPLICABILITY

According to the present disclosure, it is possible to produce a hydrocarbon having 2 or more carbon atoms with a high yield even at a high temperature under pressure and to reduce the size of a facility that performs an OCM reaction.

1. An oxidative coupling catalyst which generates a hydrocarbon having 2 or more carbon atoms from methane by an oxidative coupling reaction of methane,
wherein the oxidative coupling catalyst is a sintered substance of silica on which an alkali metal salt is supported, and has a particle diameter of 1 to 5 mm.

2. The oxidative coupling catalyst according to claim 1,
wherein the sintered substance of silica on which the alkali metal salt is supported exhibits crystallinity.

3. The oxidative coupling catalyst according to claim 1,
wherein the alkali metal salt is an alkali metal salt of an oxide containing at least one of tungsten or zirconium.

4. The oxidative coupling catalyst according to claim 3,
wherein the alkali metal salt is a potassium salt of the oxide.

5. The oxidative coupling catalyst according to claim 4,
wherein manganese is further supported.

6. The oxidative coupling catalyst according to claim 5,
wherein a mass ratio represented by manganese:potassium salt of oxide:silica is (0.01 to 5):(0.05 to 10):(85 to 99.9).

7. The oxidative coupling catalyst according to claim 5,
wherein tin is further supported.

8. The oxidative coupling catalyst according to claim 7,
wherein a mass ratio represented by tin:manganese:potassium salt of oxide:silica is (0.01 to 50):(0.01 to 5):(0.05 to 10):(35 to 99.9).

9. (canceled)

10. A method for producing an oxidative coupling catalyst which generates a hydrocarbon having 2 or more carbon atoms from methane by an oxidative coupling reaction of methane, the method comprising:
a step of bringing an aqueous solution containing an alkali metal salt into contact with silica or a sintered substance of silica to obtain a precursor in which the silica or the sintered substance of silica is impregnated with the aqueous solution; and
a step of sintering the precursor at 800° C. or higher to obtain the oxidative coupling catalyst having a particle diameter of 1 to 5 mm.

11. The method for producing an oxidative coupling catalyst according to claim 10, further comprising:
a step of extrusion-molding a composition in which silica powder is dispersed in a binder to obtain the silica.

12. The method for producing an oxidative coupling catalyst according to claim 11, further comprising:
a step of sintering a molded body obtained by extrusion-molding the composition at 700° C. or higher to obtain the sintered substance of silica.

13. The method for producing an oxidative coupling catalyst according to claim 12, further comprising:
a step of obtaining the sintered substance of silica,
wherein the sintered substance of silica has a crushing strength of 10 N/mm² or less.

14. The method for producing an oxidative coupling catalyst according to claim 10,
wherein the silica is amorphous.

15. A method for producing hydrocarbons, comprising:
generating a hydrocarbon having 2 or more carbon atoms from methane by an oxidative coupling reaction of methane using the oxidative coupling catalyst according to claim 1.

16. The method for producing hydrocarbons according to claim 15,
wherein the oxidative coupling reaction is performed under a pressurized condition.

17. The method for producing hydrocarbons according to claim 16,
wherein a pressure under the pressurized condition is 0.9 MPa or greater.

18. The method for producing hydrocarbons according to claim 17,
wherein the oxidative coupling reaction is performed at 500° C. or higher.
